(19) United States
(12) Patent Application Publication
(10) Pub. No.: US 2018/0042459 A1
(43) Pub. Date: Feb. 15, 2018

(54) MEDICAL TOOL WITH IMAGE ENHANCING FEATURES

(71) Applicant: Soundcore Technology, LLC., Mercer Island, WA (US)

(72) Inventors: Christopher Loiselle, Mercer Island, WA (US); R. Maxwell Flaherty, Auburndale, FL (US); J. Christopher Flaherty, Auburndale, FL (US)

(21) Appl. No.: 15/552,845

(22) PCT Filed: Feb. 23, 2016
(86) PCT No.: PCT/US16/19042

§ 371 Date: Aug. 23, 2017

Related U.S. Application Data
(60) Provisional application No. 62/119,395, filed on Feb. 23, 2015.

(51) Int. Cl. A61B 1/00; A61B 5/06; G06T 7/00; A61B 5/00

(52) U.S. Cl. CPC A61B 1/00087; A61B 5/0044; A61B 5/066; G06T 7/0012; A61B 1/00096; A61B 1/00009; A61B 1/00016; A61B 1/00177; A61B 1/005

(57) ABSTRACT

A medical tool comprises: an elongate shaft comprising a wall and at least one inner core. The shaft is constructed and arranged to perform an image enhancing function selected from the group consisting of: minimize image distortion caused by the medical tool; reduce image scatter caused by the medical tool; reduce image noise caused by the medical tool; reduce image degradation caused by the medical tool; enhance visualization of the medical tool within the medical image; and combinations thereof. Systems comprising the medical tool and an imaging device are also provided.

### RELATED APPLICATIONS

This application claims the benefit of U.S. Provisional Application Ser. No. 62/119,395, titled “Medical Tool with Image Enhancing Features”, filed Feb. 23, 2015, the content of which is incorporated herein by reference in its entirety for all purposes.

### FIELD OF THE INVENTION

Inventive concepts relate generally to medical tools for performing a procedure on a patient, and in particular, tools that include features constructed and arranged to enhance an image.

### BACKGROUND

Various forms of medical imaging are increasingly used during medical and surgical procedures today. Unfortunately, many medical and surgical instruments degrade medical imaging quality. There is a need for medical tools that improve imaging quality and/or reduce adverse effects caused by the tools, such as to provide increased effectiveness of procedures using medical and surgical instruments, reduce procedural complications, and overall improve care of patients.

### SUMMARY

Described herein are medical tools for treating a patient and/or gathering patient information. According to one aspect of the present inventive concepts, a medical tool comprises an elongate shaft comprising a wall and at least one inner core. The shaft is constructed and arranged to perform an image enhancing function selected from the group consisting of: minimize image distortion caused by the medical tool; reduce image scatter caused by the medical tool; reduce image noise caused by the medical tool; reduce image degradation caused by the medical tool; enhance visualization of the medical tool within a medical image; and combinations thereof.

In some embodiments, the medical tool comprises a therapeutic tool.

In some embodiments, the medical tool is constructed and arranged to gather patient information.

In some embodiments, the medical tool comprises a diagnostic tool.

In some embodiments, the medical tool comprises a uterine sound.

In some embodiments, the medical tool comprises a dilator.

In some embodiments, the medical tool comprises scissors.

In some embodiments, the medical tool comprises a probe.

In some embodiments, the medical tool comprises a manipulator.

In some embodiments, the medical tool comprises a curette.

In some embodiments, the medical tool comprises forceps.

In some embodiments, the medical tool comprises a tissue scraper.

In some embodiments, the medical tool comprises a tissue grasping device.

In some embodiments, the medical tool comprises a tunneling device.

In some embodiments, the medical tool comprises a stylet.

In some embodiments, the medical tool comprises a brachytherapy tool.

In some embodiments, the medical tool comprises a radioactive source placement tool.

In some embodiments, the medical tool comprises an electrical energy delivery device.

In some embodiments, the medical tool comprises a loop cautery device.

In some embodiments, the medical tool comprises a suction device.

In some embodiments, the medical tool is constructed and arranged to perform a cardiac procedure. The medical tool can be configured to be guided by an imaging device selected from the group consisting of: ultrasonic imaging device; MRI; and combinations thereof.

In some embodiments, the medical tool comprises a robotically assisted tool.

In some embodiments, the medical tool comprises at least a portion configured to be positioned at least 10 cm below a surface of a patient's skin during creation of a medical image. The medical image can be produced by an ultrasound imaging device.

In some embodiments, the medical tool comprises at least a portion configured to be positioned between 10 cm and 14 cm below a surface of a patient's skin during creation of a medical image. The medical image can be produced by an ultrasound imaging device.

In some embodiments, the medical tool comprises a sterile tool. The medical tool can be constructed and arranged to be re-sterilized.

In some embodiments, the medical tool comprises a single-use tool.

In some embodiments, the medical tool comprises a multiple use tool.

In some embodiments, the medical image comprises an image produced by an ultrasound imaging device. The ultrasound imaging device can comprise an operating frequency between 2 MHz and 5 MHz.

In some embodiments, the medical image comprises an image produced by an MRI.

In some embodiments, the medical image comprises an image produced by a CT-Scanner.

In some embodiments, the medical image comprises an image produced by an imaging device selected from the group consisting of: ultrasound imaging device; 2D ultrasound imaging device; 3D ultrasound imaging device; MRI; CT-Scanner; X-ray imager; fluoroscope; nuclear medical scanner; and combinations thereof.

In some embodiments, the elongate shaft comprises a length of at least 3 inches. The elongate shaft can comprise a length of at least 6 inches. The elongate shaft can comprise a length of approximately 9 inches. The elongate shaft can comprise a length of at least 24 inches. The medical tool can comprise a laparoscopic tool.

In some embodiments, the elongate shaft comprises at least a flexible portion.

In some embodiments, the elongate shaft comprises at least a malleable portion.

In some embodiments, the elongate shaft comprises at least a rigid portion.

In some embodiments, the elongate shaft comprises a diameter of at least 1.5 mm. The elongate shaft can comprise a diameter of at least 2.5 mm.

In some embodiments, the elongate shaft comprises a diameter between 1.5 mm and 25 mm. The elongate shaft can comprise a diameter between 2.5 mm and 10 mm.

In some embodiments, the medical tool is constructed and arranged to provide shaft orientation information on the medical image. The shaft orientation information can comprise at least one of: translational position of the shaft or rotational position of the shaft. The wall can comprise a circular cross section and the at least one inner core can have a non-circular cross section constructed and arranged to provide the shaft orientation information. The shaft can comprise an orientation marker. The orientation marker can comprise a marker selected from the group consisting of: ultrasonically reflective marker; radiopaque marker; magnetic marker; and combinations thereof. The shaft can comprise an eccentric cross sectional geometry constructed and arranged to provide the shaft orientation information.

In some embodiments, the shaft comprises a single shaft.

In some embodiments, the shaft comprises multiple shafts.

In some embodiments, the medical tool further comprises a filament positioned in the shaft. The filament can comprise a filament selected from the group consisting of: electrical wire; control rod; optical fiber; fluid delivery tube; and combinations thereof.

In some embodiments, the shaft comprises at least one lumen. The lumen can comprise a fluid delivery lumen. The medical tool can further comprise a filament positioned in the shaft. The filament can comprise a filament selected from the group consisting of: electrical wire; control rod; optical fiber; fluid delivery tube; and combinations thereof.

In some embodiments, the shaft comprises multiple lumens. The multiple lumens can be arranged in a pattern selected from the group consisting of: concentric; non-concentric; adjacent; overlapping; non-overlapping; and combinations thereof.

In some embodiments, the shaft comprises a relatively constant diameter along its length.

In some embodiments, the shaft comprises a relatively similar cross sectional geometry along its length.

In some embodiments, the shaft comprises a varying diameter along its length.

In some embodiments, the shaft comprises a varying cross sectional geometry along its length.

In some embodiments, the shaft comprises a first end with a first diameter, and a second end with a second diameter different than the first diameter.

In some embodiments, the shaft comprises a non-linear construction. The shaft can comprise a mid-portion with a first axis and a distal portion with a second axis, and the second axis can be angularly offset from the first axis. The second axis can be angularly offset from the first axis with an angle between 5° and 60°.

In some embodiments, the wall is constructed and arranged to reduce distortion of the medical image.

In some embodiments, the wall is constructed and arranged to enhance imaging of tissue proximate the wall in the medical image.

In some embodiments, the wall comprises a tubular construction.

In some embodiments, the wall comprises a material selected from the group consisting of: one or more metals; stainless steel; titanium; shaped memory alloy; nickel titanium alloy; one or more plastics; carbon fiber; polyvinyl chloride (PVC); silicone; one or more thermoplastic elastomers; one or more polymers; and combinations of one or more of these.

In some embodiments, the wall comprises carbon fiber.

In some embodiments, the wall comprises a flexible titanium tube.

In some embodiments, the wall comprises a metal. The wall can comprise stainless steel. The wall can comprise titanium. The wall can comprise shaped memory alloy. The wall can comprise nickel titanium alloy.

In some embodiments, the wall comprises plastic. The medical image can comprise an MRI image.

In some embodiments, the wall comprises a thickness constructed and arranged to reduce distortion of a medical image. The wall can comprise a thickness less than 3.0 mm. The wall can comprise a thickness less than 2.5 mm. The wall can comprise a thickness less than 2.0 mm. The wall can comprise a thickness less than 1.8 mm. The wall can comprise a thickness less than 1.6 mm. The wall can comprise a thickness less than 1.4 mm. The wall can comprise a thickness less than 1.2 mm. The wall can comprise a thickness less than 1.0 mm. The wall can comprise a metal material.

In some embodiments, the shaft comprises a diameter of approximately 2.8 mm and the wall comprises a thickness of approximately 0.5 mm.

In some embodiments, the shaft comprises a diameter of approximately 2.8 mm and the wall comprises a thickness of less than or equal to 0.5 mm.

In some embodiments, the wall comprises a thickness between 0.63 mm and 1.08 mm. The shaft can comprise a diameter between 3.96 mm and 4.06 mm.

In some embodiments, the wall comprises a thickness of approximately 0.17 mm. The shaft can comprise a diameter between 2.76 mm and 5.56 mm. The medical tool can comprise a single-use product.

In some embodiments, the wall comprises a thickness between 0.17 mm and 0.38 mm. The shaft can comprise a diameter between 3.04 mm and 5.56 mm.

In some embodiments, the wall comprises a thickness between 0.25 mm and 0.76 mm.

In some embodiments, the wall comprises a thickness of approximately 0.33 mm.

In some embodiments, the wall comprises a thickness between 0.25 mm and 0.60 mm.

In some embodiments, the wall comprises a thickness between 0.25 mm and 0.38 mm.

In some embodiments, the wall comprises a thickness between 1% and 20% of the diameter of the shaft. The wall can comprise a thickness between 1% and 15% of the diameter of the shaft. The wall can comprise a thickness approximately 5% of the diameter of the shaft.

In some embodiments, the wall is constructed and arranged to be adjusted to modify the image enhancing function. The wall can comprise an adjustable diameter. The wall can be configured to at least one of: furl or unfurl. The at least one inner core can comprise an adjustable diameter. The at least one inner core can be constructed and arranged to be adjusted to modify the image enhancing function.

In some embodiments, the wall comprises an outer surface along its entire length, and an inner surface along at least a portion of its length. The wall outer surface can comprise at least one circular shaped cross section. The wall inner surface can comprise a cross sectional shape selected from the group consisting of: circular; elliptical; rectangular; trapezoidal; regular polygon; irregular polygon; spiral; and combinations thereof, within the at least one circular shaped cross section of the outer surface. The wall outer surface can comprise at least one elliptical shaped cross section. The wall inner surface can comprise a cross sectional shape selected from the group consisting of: circular; elliptical; rectangular; trapezoidal; regular polygon; irregular polygon; spiral; and combinations thereof, within the at least one elliptical shaped cross section of the outer surface. The wall outer surface can comprise at least one rectangular shaped cross section. The wall inner surface can comprise a cross sectional shape selected from the group consisting of: circular; elliptical; rectangular; trapezoidal; regular polygon; irregular polygon; spiral; and combinations thereof, within the at least one rectangular shaped cross section of the outer surface. The wall outer surface can comprise at least one trapezoidal shaped cross section. The wall inner surface can comprise a cross sectional shape selected from the group consisting of: circular; elliptical; rectangular; trapezoidal; regular polygon; irregular polygon; spiral; and combinations thereof, within the at least one trapezoidal shaped cross section of the outer surface. The wall outer surface can comprise a varying cross sectional geometry along the length of the wall. The wall outer surface varying cross sectional geometry can comprise a tapered geometry. The wall outer surface varying cross sectional geometry can comprise a varying cross sectional shape. The at least one inner core can comprise an outer surface defined by the wall inner surface. The wall inner surface can comprise a varying cross section along the at least a portion of the length of the wall. The wall inner surface varying cross sectional geometry can comprise a tapered geometry. The wall inner surface varying cross sectional geometry can comprise a varying cross sectional shape. The wall inner surface can comprise at least one cross section with a geometry selected from the group consisting of: circular; elliptical; rectangular; trapezoidal; regular polygon; irregular polygon; spiral; and combinations thereof. The wall outer surface can comprise at least cross section with a geometry selected from the group consisting of: circular; elliptical; rectangular; trapezoidal; regular polygon; irregular polygon; spiral; and combinations thereof. The wall inner surface can comprise a first cross sectional geometry, and the outer surface can comprise a second cross sectional geometry, and the first cross sectional geometry can be different than the second cross sectional geometry.

In some embodiments, the at least one inner core comprises a material with a density approximating the density of fat tissue.

In some embodiments, the at least one inner core comprises a material selected from the group consisting of: a solid material; a fluid; a liquid material; water; saline; a gas; plastic; elastomer; gel; hydrogel; foam; glass; silicone; polyvinyl chloride; a polymer; and combinations thereof.

In some embodiments, the at least one inner core comprises a gel. The gel can comprise ultrasound gel.

In some embodiments, the at least one inner core comprises at least one of a gas or a vacuum, and the medical tool is configured to be imaged by an ultrasound imaging device.

In some embodiments, the at least one inner core comprises at least one of a liquid or a solid, and the medical tool is configured to be imaged by an MRI.

In some embodiments, the at least one inner core comprises a metal-impregnated material. The at least one inner core can comprise a metal-impregnated material selected from the group consisting of: a metal-impregnated solid; a metal-impregnated liquid; a metal-impregnated gel; a metal-impregnated foam; and combinations thereof.

In some embodiments, at least one inner core comprises a hollow segment of the shaft. The at least one inner core can further comprise a fluid. The fluid can comprise a material selected from the group consisting of: liquid; gas; gel; and combinations thereof. The fluid can comprise a material selected from the group consisting of: air; carbon dioxide; nitrogen; and combinations thereof. The at least one inner core can comprise at least one of a partial vacuum or a complete vacuum. The at least one inner core can further comprise a material comprising multiple gas bubbles. The at least one inner core can comprise a foam material. The at least one inner core can further comprise a material with a density approximating the density of fat tissue.

In some embodiments, the at least one inner core is positioned along a majority of the length of the shaft.

In some embodiments, the at least one inner core is positioned within a distal portion of the shaft.

In some embodiments, the at least one inner core is constructed and arranged to reduce distortion of a medical image.

In some embodiments, the at least one inner core comprises a relatively continuous cross section along the length of the at least one inner core.

In some embodiments, the at least one inner core comprises a varying cross section along the length of the at least one inner core. The at least one inner core can comprise a varying cross sectional area along the length of the at least one inner core. The at least one inner core can comprise a varying cross sectional shape along the length of the at least one inner core.

In some embodiments, the at least one inner core comprises a translating shaft.

In some embodiments, the medical tool further comprising a functional element positioned on the shaft, and the at least one inner core comprises a conduit operably attached to the functional element. The functional element can comprise an element selected from the group consisting of: probe; tunneling tip; grasper; cutter; drug delivery element; electrode; energy delivery element; RF energy delivery element; radiation delivery element; and combinations thereof.

In some embodiments, the at least one inner core is constructed and arranged to be adjusted to modify the image enhancing function. The at least one inner core can comprise an adjustable diameter. The medical tool can be constructed and arranged to adjust the diameter by adjusting the pressure of the at least one inner core. The medical tool can be constructed and arranged to adjust the diameter by adjusting the volume of the at least one inner core. The at least one inner core can comprise an adjustable density. The medical tool can be constructed and arranged to adjust the density by adjusting the pressure of the at least one inner core. The at least one inner core can comprise an adjustable volume. The medical tool can comprise a handle including a port, and the volume of the at least one inner core can be adjusted by at least one of adding or removing material to or from the at least one inner core via the port. The at least one inner core can comprise an adjustable material type. The medical tool can comprise a handle including a port, and the material type of the at least one inner core can be adjusted by at least one of adding or removing material to or from the at least one inner core via the port.

In some embodiments, the at least one inner core is configured to be removed from the shaft by an operator of the medical tool. The at least one inner core removed from the shaft by an operator of the medical tool can comprise a gas.

In some embodiments, the at least one inner core is configured to be introduced into the shaft by an operator of the medical tool. The at least one inner core introduced into the shaft by an operator can comprise at least one of a liquid or a solid. The medical tool can be configured to be imaged by an MRI.

In some embodiments, the medical tool further comprises an access port constructed and arranged to allow material to be at least one of introduced into or removed from the shaft. The material can comprise the inner core. The access port can comprise a luer connector. The access port can be configured to allow an operator to change the inner core from a first material to a second material. The first material can comprise a gas and the second material can comprise at least one of a liquid or a solid.

In some embodiments, the shaft comprises a proximal end, and the medical tool further comprises a handle positioned on the proximal end of the shaft. The medical tool can further comprise a control positioned on the handle. The medical tool can further comprise a functional element positioned on the shaft, and the control can be configured to modify the function of the functional element.

In some embodiments, the wall comprises an outer surface along its length, and the shaft comprises a coating on at least a portion of the wall outer surface. The coating can comprise an ultrasonically reflective coating. The coating can comprise a radiopaque coating. The coating can comprise a magnetic coating.

In some embodiments, the medical tool further comprises a functional element positioned on the shaft. The shaft can comprise a distal portion, and the functional element can be positioned on the shaft distal portion. The shaft can comprise a distal end, and the functional element can be positioned on the shaft distal end. The shaft can comprise an outer surface, and the functional element can be positioned on the shaft outer surface. The functional element can comprise an element selected from the group consisting of: probe; tunneling tip; grasper; cutter; agent delivery element; electrode; energy delivery element; RF energy delivery element; cautery element; desiccating element; and combinations thereof. The functional element can comprise a probe tip. The functional element can comprise a blunt tip. The functional element can comprise a tunneling tip. The functional element can comprise an energy delivery element. The energy delivery element can be configured to deliver an energy selected from the group consisting of: electromagnetic energy; radiofrequency energy; microwave energy; light energy; laser light energy; sound energy; ultrasonic sound energy; subsonic sound energy; thermal energy; heat energy; cryogenic energy; infrared energy; terahertz energy; ultraviolet energy; visible light energy; and combinations thereof. The functional element can comprise an agent delivery element. The agent delivery element can be constructed and arranged to deliver an agent selected from the group consisting of: a drug; a chemotherapeutic agent; an agent configured for biologically targeted therapy; an immunotherapy agent; and combinations thereof. The functional element can comprise a radiation delivery element. The radiation delivery element can be constructed and arranged to deliver a radiation source selected from the group consisting of: radioactive seeds; radionuclide agent; and combinations thereof. The medical tool can further comprise an image enhancing element positioned within the functional element.

According to another aspect of the present inventive concepts, a medical system comprises a medical tool of the present inventive concepts as described herein, and an imaging device.

In some embodiments, the imaging device comprises an ultrasonic imaging device.

In some embodiments, the imaging device comprises an MRI.

In some embodiments, the imaging device comprises a CT-scanner.

In some embodiments, the imaging device comprises an X-ray imager.

In some embodiments, the imaging device comprises a Fluoroscope.

In some embodiments, the medical system further comprises a body introduction device, and the medical tool is configured to pass through the body introduction device and into the patient. The body introduction device can comprise a device selected from the group consisting of: speculum; a vascular introducer; a laparoscopic port; and combinations thereof. The body introduction device can comprise a vaginal speculum.

In some embodiments, the medical tool comprises a first medical tool, and the medical system further comprises a second medical tool as described herein. The first medical tool and the second medical tool can comprise different characteristics. The different characteristics can comprise different wall thicknesses. The first medical tool and the second medical tool can comprise similar shaft diameters. The different characteristics can comprise different shaft diameters. The medical system can comprises a kit of medical tools with shaft diameters selected from the group consisting of: 1.5 mm; 2.0 mm; 2.5 mm; 5.0 mm; 7.5 mm; 10.0 mm and/or 12.5 mm. The medical system can comprise a kit of medical tools with at least two shaft diameters selected from the group consisting of: 1.5 mm; 2.0 mm; 2.5 mm; 5.0 mm; 7.5 mm; 10.0 mm; and 12.5 mm. The medical system can comprise a kit of medical tools with at least two shaft diameters ranging from 1.5 mm and 6.0 mm. The medical system can comprise a kit of medical tools with at least two shaft diameters ranging from 2.0 mm and 5.0 mm. The medical system can comprise a kit of medical tools with at least two shaft diameters ranging from 3.0 mm and 5.0 mm. The medical system can comprise a kit of medical tools comprising shaft diameters of 3.0 mm, 4.0 mm and 5.0 mm. The kit can comprise three medical tools. The kit can comprise less than three medical tools, and at least one medical tool can comprise a double ended shaft. The medical system can comprise a kit of medical tools comprising shaft diameters of 2.0 mm, 3.0 mm, 4.0 mm, 5.0 mm and 6.0 mm. The kit can comprise five medical tools. The kit can comprise less than five medical tools, and at least one medical tool can comprise a double-ended shaft.

According to another aspect of the present inventive concepts, a method of using the medical tool of the present inventive concepts is provided. The method can comprise selecting a medical tool; introducing the medical tool into the patient; and creating a medical image.

In some embodiments, multiple images medical are created while adjusting at least one of the shaft wall or the inner shaft portion.

In some embodiments, the method further comprises delivering the at least one inner core within the shaft. The delivering of the at least one inner core within the shaft can occur prior to creating the medical image.

In some embodiments, the method further comprises modifying the inner core.

The modification can comprise changing the inner core from a gas to at least one of a liquid or a solid.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view and a magnified side sectional view of a system including a medical tool comprising an image enhancing feature, consistent with the present inventive concepts.

FIG. 2A is a sectional side view of the distal portion of a medical tool comprising a functional element at its tip and three image enhancing features, consistent with the present inventive concepts.

FIG. 2B is a sectional side view of the distal portion of a medical tool comprising a functional element at its tip and four image enhancing features, consistent with the present inventive concepts.

FIG. 3A is a side view and a magnified side sectional view of a uterine sound tool comprising an image enhancing feature, consistent with the present inventive concepts.

FIG. 3B is a side view and a magnified side sectional view of a scraping tool comprising an image enhancing feature, consistent with the present inventive concepts.

FIG. 3C is a side view and a magnified side sectional view of forceps comprising an image enhancing feature, consistent with the present inventive concepts.

FIG. 3D is a side view and a magnified side sectional view of a tissue grasping tool comprising an image enhancing feature and a translating inner portion, consistent with the present inventive concepts.

FIG. 3E is a schematic view and a magnified side sectional view of a system including a medical tool comprising an image enhancing feature and a camera, consistent with the present inventive concepts.

FIG. 3F is a side view, a magnified side sectional view and an end sectional view of a medical tool comprising an image enhancing feature and a dual lumen shaft, consistent with the present inventive concepts.

FIG. 3G is a side view, a magnified side sectional view and a magnified end sectional view of a medical tool comprising an image enhancing feature and a translating shaft, consistent with the present inventive concepts.

FIGS. 4A-4P are end sectional views of various medical tool shafts comprising different cross sectional geometries constructed and arranged to enhance an image, consistent with the present inventive concepts.

FIGS. 5A and 5B are recreations of an ultrasound image, a sagittal view and an axial view, respectively, of the uterus of a patient into which a standard tool has been positioned.

FIGS. 6A and 6B are recreations of an ultrasound image, a sagittal view and an axial view, respectively, of the uterus of a patient into which a medical tool of the present inventive concepts has been positioned.

FIG. 7 is an end sectional view of a shaft of a medical tool comprising an adjustable diameter wall, consistent with the present inventive concepts.

FIG. 7A is an end sectional view of the shaft of FIG. 7, after increasing the diameter of the shaft, consistent with the present inventive concepts.

FIG. 8 is a side view and a magnified side sectional view of a medical tool comprising an adjustable inner core, consistent with the present inventive concepts.

FIG. 9 is a side view and a magnified side sectional view of a medical tool comprising an adjustable image enhancing feature, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be tended a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

The present inventive concepts include medical tools that include one or more image enhancing features constructed and arranged to enhance a medical image, such as to prevent or at least reduce (hereinafter “reduce”) distortion of a medical image and/or enhance imaging of the medical tool in the medical image. These medical tools can be constructed and arranged to perform a therapeutic procedure and/or to gather patient data, such as in a diagnostic procedure. Each medical tool can comprise a shaft or other portion that includes one or more image enhancing features, such as a feature constructed and arranged to perform an image enhancing function selected from the group consisting of: minimize image distortion caused by the medical tool; reduce image scatter caused by the medical tool; reduce image noise caused by the medical tool; reduce image degradation caused by the medical tool; enhance visualization of the medical tool within the medical image; and combinations of one or more of these.

The medical tools of the present inventive concepts can be constructed and arranged to allow surgeons, other physicians, and other medical staff to perform real-time image guided surgeries or other medical procedures with better effectiveness and lower risk of complications. The medical tools of the present inventive concepts have enhanced compatibility with imaging devices such as ultrasound imagers (e.g. 2D or 3D ultrasound imagers), CAT (CT) scanners, and magnetic resonance imaging devices (MRI). In some embodiments, a medical tool comprises a uterine sound device constructed and arranged to avoid scattering of imaging energy, such as ultrasound waves, and provides both a crisp image of the device and the patient's tissue proximate, behind, and/or lateral to the device. In some embodiments, a medical tool comprises a tool used in a cardiac procedure, such as a cardiac procedure in which the tool is guided using ultrasound and/or MRI.

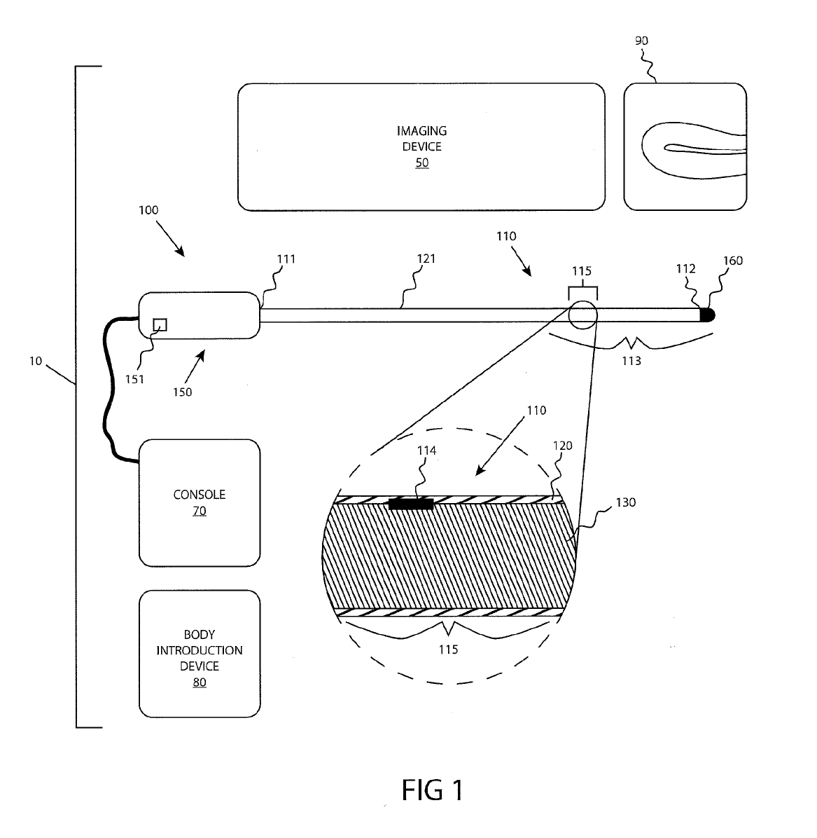

Referring now to FIG. 1, a schematic view and a magnified side sectional view of a system including a medical tool comprising an image enhancing feature are illustrated, consistent with the present inventive concepts. System 10 comprises medical tool 100 and imaging device 50. Imaging device 50 can comprise one or more imaging devices constructed and arranged to create medical image 90 comprising one or more images of a portion of a patient and any tools positioned within that patient portion. In some embodiments, imaging device 50 comprises an imaging device selected from the group consisting of: ultrasound imager; magnetic resonance imager (MRI); CT-Scanner; X-ray; fluoroscope; nuclear medical scan; and combinations of one or more of these. In some embodiments, medical image 90 comprises one or more ultrasound images such as is described herebelow in reference to FIGS. 6A-B. In some embodiments, imaging device 50 comprises an ultrasound imaging device operating at a frequency between 2 MHz and 5 MHz.

In some embodiments, medical tool 100 is operably attached to a console 70, such as a console configured to deliver energy and/or an agent to medical tool 100 as described herebelow. Medical tool 100 comprises shaft 110, which includes proximal end 111, distal portion 113 and distal end 112. A handle 150 is positioned on proximal end 111. Handle 150 can comprise one or more controls, such as control 151 shown. Medical tool 100 can comprise one or more functional elements, such as functional element 160 positioned on distal end 112 of shaft 110, and described in detail herebelow. In some embodiments, system 10 comprises body introduction device 80 through which medical tool 100 is introduced into a patient. Body introduction device 80 can comprise a one, two, or more of: a speculum (e.g. a vaginal speculum), a vascular introducer, a laparoscopic port, or other body introduction device.

Medical tool 100 can comprise one or more features constructed and arranged to enhance a medical image, such as medical image 90 produced by imaging device 50. For example, as shown in the magnified view of FIG. 1, a portion of shaft 110 positioned in distal portion 113, segment 115, comprises wall 120 and inner core 130. Wall 120 can comprise a tubular construction, such as a hollow tube into which inner core 130 is positioned. Wall 120 and/or inner core 130 are constructed and arranged to enhance a medical image, such as medical image 90. Wall 120 and/or inner core 130 can be constructed and arranged to reduce distortion of the medical image and/or enhance imaging of tissue proximate wall 120. For example, wall 120 can comprise materials and/or a thickness constructed and arranged to reduce scattering and/or any undesired image-impacting effect upon an imaging signal (e.g. a magnetic field transmitted by an MRI; an ultrasound signal transmitted by an ultrasonic imaging device; an X-ray transmitted by a fluoroscope, CT scanner, and/or other X-ray imager; and combinations of one or more of these). In some embodiments, at least a portion of medical tool 100 (e.g. distal portion 112 or another portion of shaft 110) is configured to be positioned at least 10 cm below the surface of the patient's skin (e.g. when medical image 90 is being created), such as between 10 cm and 14 cm below the patient's skin, such as when imaging device 50 comprises an ultrasound imager or other imaging device.

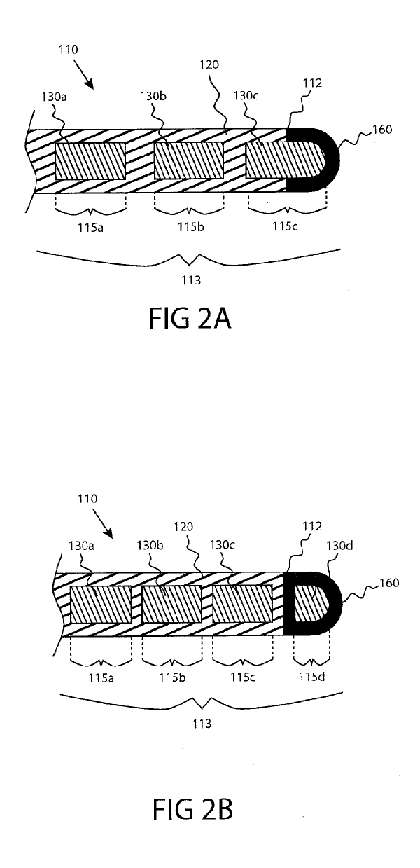

Inner core 130 can comprise a single inner core 130 that is positioned along a majority of the length of shaft 110 (e.g. along a majority of the length of wall 120). Alternatively, inner core 130 can comprise one or more smaller segments positioned within one or more portions of wall 120, such as is described herebelow in reference to FIG. 2A or 2B.

Wall 120 can comprise a flexible portion, a rigid portion and/or a malleable portion. Wall 120 can comprise one or more metallic or non-metallic materials. In some embodiments, wall 120 comprises a material selected from the group consisting of: one or more metals; stainless steel; titanium; shaped memory alloy; nickel titanium alloy; one or more plastics; carbon fiber; polyvinyl chloride (PVC); silicone; one or more thermoplastic elastomers; one or more polymers; and combinations of one or more of these. In some embodiments, wall 120 comprises a flexible titanium tube. In some embodiments, wall 120 comprises a carbon fiber, such as when imaging device comprises an MRI.

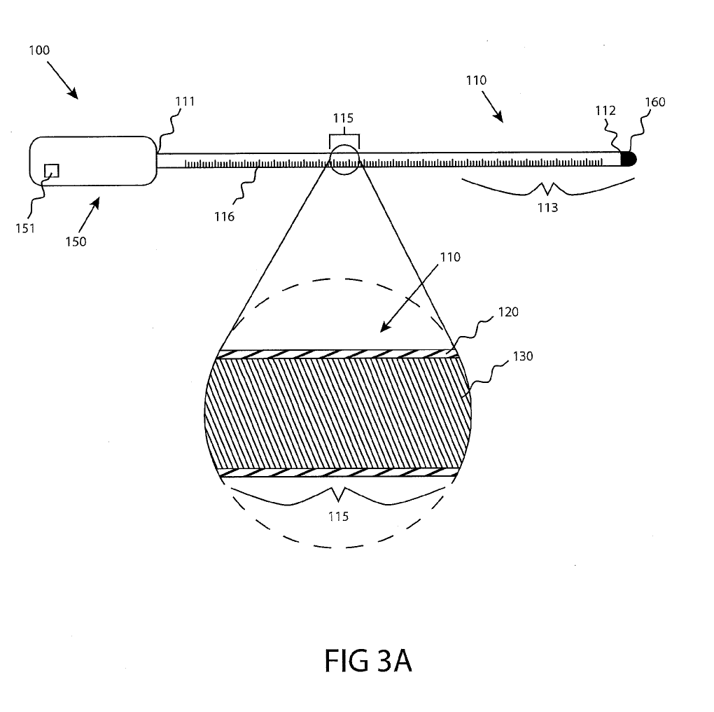

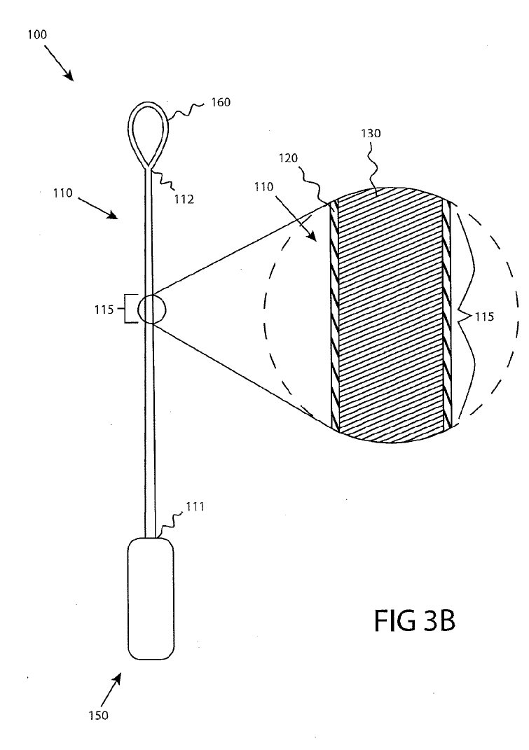

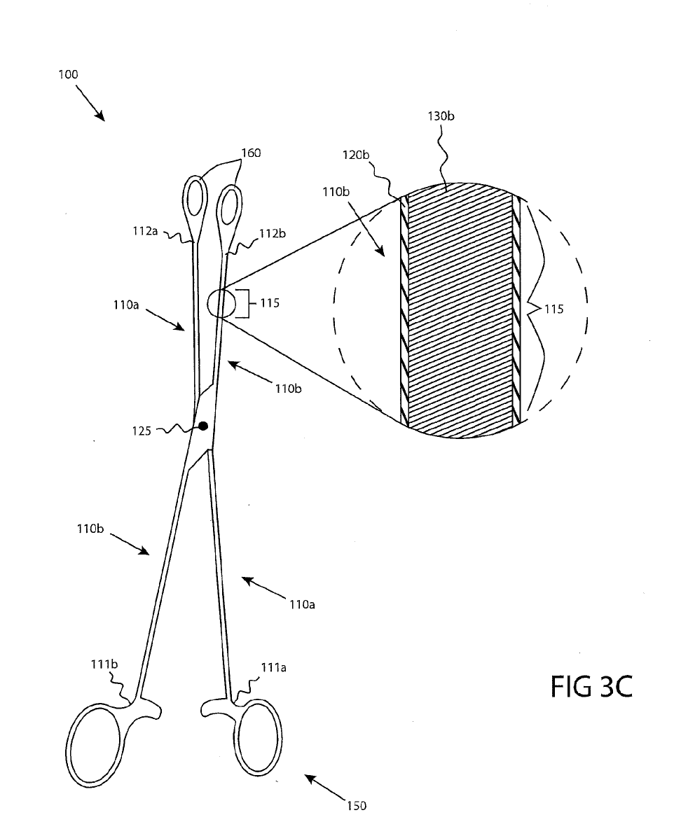

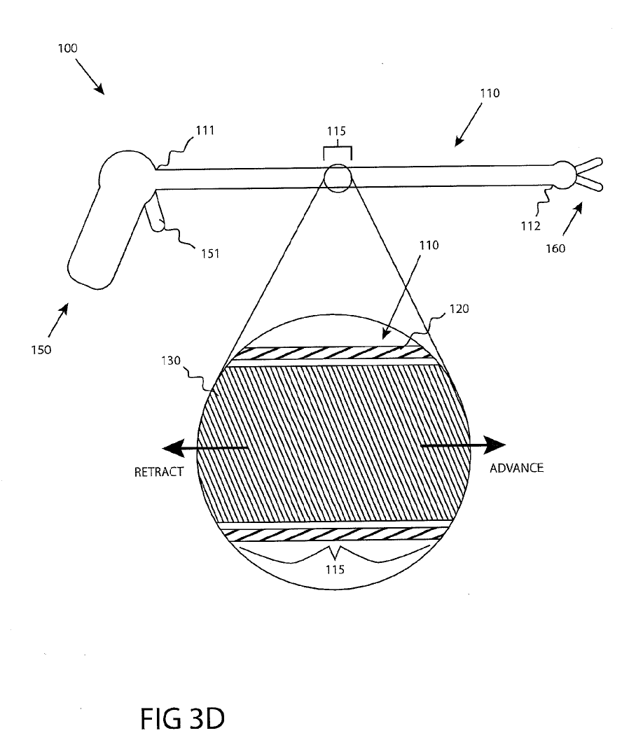

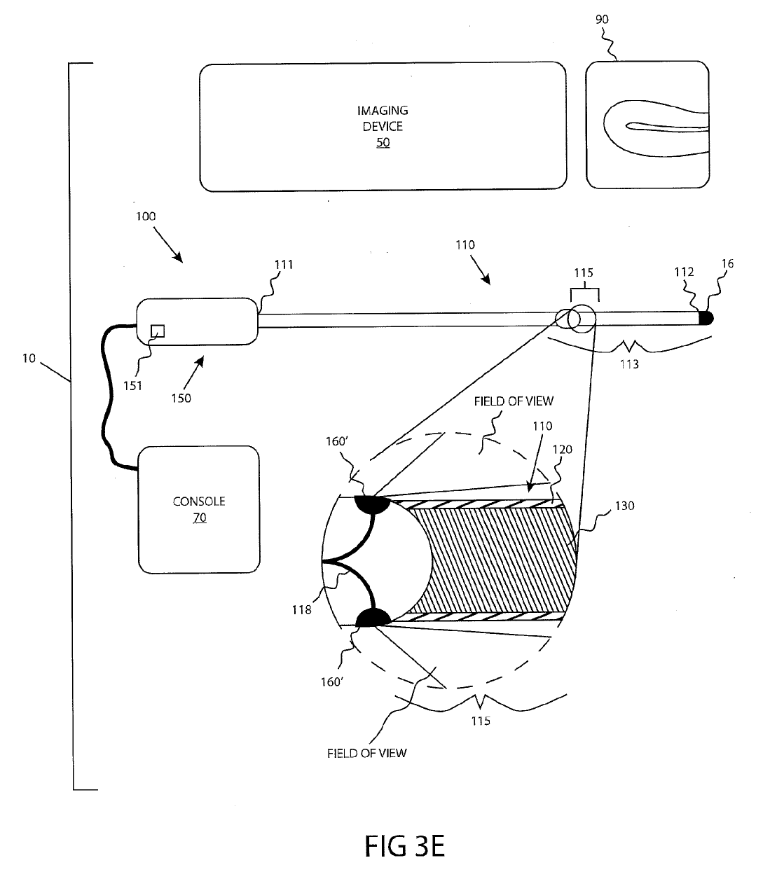

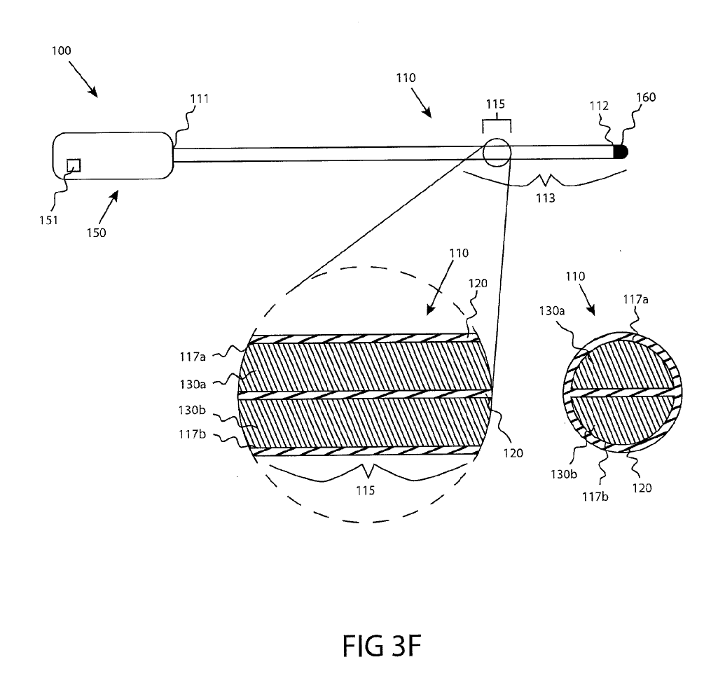

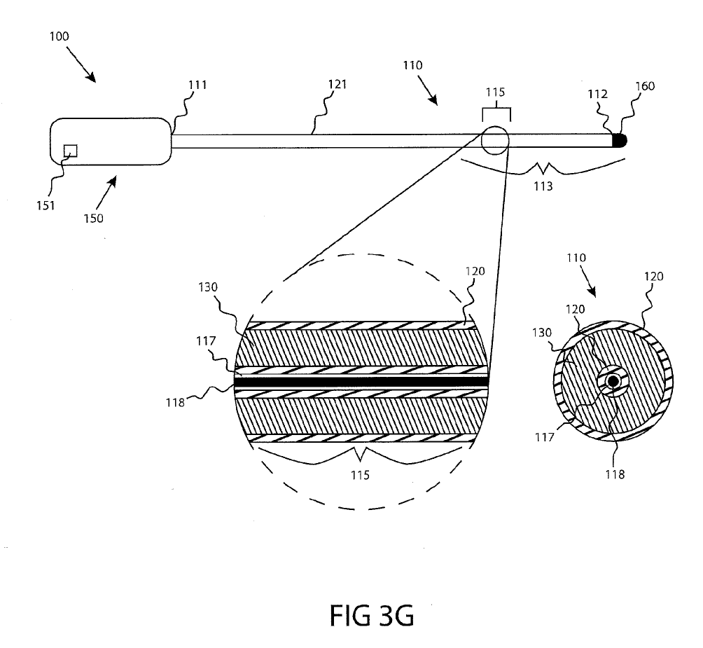

Inner core 130 can comprise a flexible portion, a rigid portion and/or a malleable portion. In some embodiments, inner core 130 is translatable (e.g. advanceable or retractable) within shaft 110, such as via control 151, such as is described herebelow in reference to FIG. 3D. In some embodiments, inner core 130 is operably attached to functional element 160, such as when functional element 160 comprises an element selected from the group consisting of: probe; tunneling tip; grasper; cutter; drug delivery element; electrode; energy delivery element; RF energy delivery element; radiation delivery element; and combinations of one or more of these. Inner core 130 can comprise: one or more wires, optical fibers or other conduits configured to provide energy to functional element 160; a translatable shaft configured to translate functional element 160; a translatable shaft configured to adjust functional element 160 (e.g. when functional element comprises scissors, grasper or any assembly comprising a portion adjustable by an applied force delivered by a shaft); one or more fluid delivery conduits such as to provide a hydraulic or pneumatic fluid; and combinations of one or more of these. In some embodiments, inner core 130 is removable from shaft 110, such as via a port on handle 150. Inner core 130 can comprise one or more metallic or non-metallic materials. In some embodiments, inner core 130 comprises a metal-impregnated material (e.g. a metal impregnated solid, gel or other liquid, or foam). In some embodiments, wall 120 comprises a metal material and inner core 130 comprises a non-metal material. In some embodiments, inner core 130 comprises a material whose density approximates the density of fat tissue. Inner core 130 can comprise a material selected from the group consisting of: a solid material; a fluid; a liquid material such as water, saline, a gel and/or other fluid; a gas; plastic; elastomer; gel; hydrogel; foam; glass; silicone; polyvinyl chloride; a polymer; and combinations of one or more of these. In some embodiments, inner core 130 comprises a gel such as ultrasound gel (e.g. Aquasonic 100 ultrasonic gel or equivalent). In some embodiments, inner core 130 can comprise a metallic solid material and/or a non-metallic solid material. In some embodiments, inner core 130 comprises a gas selected from the group consisting of: air; carbon dioxide; nitrogen; and combinations of one or more of these. In some embodiments, inner core 130 comprises a vacuum (e.g. a full or partial vacuum). In some embodiments, inner core 130 comprises a material including gas bubbles, such as a foam material, or a fluid and gas combination. In some embodiments, inner core 130 comprises a gas and/or vacuum (“gas” herein), and medical tool 100 is configured for imaging by an imaging device 50 comprising an ultrasound imager. Alternatively, inner core 130 can comprise a liquid and/or solid material, and medical tool 100 can be configured for imaging by an imaging device 50 comprising a non-ultrasound imager, such as an MRI. In some embodiments, inner core 130 can be modified and/or replaced by an operator (e.g. when a gas is replaced with a non-gas for visualization of medical tool 100 under MRI), such as is described herebelow in reference to FIG. 9.

In some embodiments, wall 120 comprises a metal, inner core 130 comprises a non-metal, and wall 120 comprises a thickness to reduce image scatter, such as a thickness less than 3.0 mm, such as a thickness less than 2.5 mm, less than 2.0 mm, less than 1.8 mm, less than 1.6 mm, less than 1.4 mm, less than 1.2 mm, or less than 1.0 mm. Alternatively or additionally, inner core 130 can comprise a material and/or dimensions constructed and arranged to reduce scattering and/or any undesired image-impacting effect upon an imaging signal. In some embodiments, wall 120 comprises a metal, and inner core 130 comprises a different metal. For example, inner core 130 can comprise a metal-impregnated material as described hereabove.

In some embodiments, shaft 110 comprises a diameter of approximately 2.8 mm and wall 120 comprises a thickness of approximately 0.5 mm or no more than 0.5 mm

In some embodiments, wall 120 comprises a thickness approximately between 0.63 mm to 1.08 mm. In these embodiments, shaft 110 can comprise a diameter approximately between 3.96 mm and 4.06 mm.

In some embodiments, wall 120 comprises a thickness of approximately 0.17 mm. In these embodiments, shaft 110 can comprise a diameter approximately between 2.76 mm to 5.56 mm. In these embodiments, medical tool 100 can comprise a single-use tool (i.e. disposed of after use in a single clinical procedure).

In some embodiments, wall 120 comprises a thickness of approximately between 0.17 mm to 0.38 mm. In these embodiments, shaft 110 can comprise a diameter approximately between 3.04 mm and 5.56 mm.

In some embodiments, wall 120 comprises a thickness of approximately between 0.25 mm to 0.76 mm.

In some embodiments, wall 120 comprises a thickness of approximately 0.33 mm, approximately between 0.25 mm and 0.60 mm, or approximately between 0.25 mm and 0.38 mm.

In some embodiments, wall 120 comprises a thickness that is between 1% and 20% of the diameter of shaft 110, such as a thickness between 1% and 15% of the diameter of shaft 110 or approximately 5% of the diameter of shaft 110.

Shaft 110 can comprise one or more elongated shafts (singly or collectively shaft 110). In some embodiments, shaft 110 comprises a relatively constant diameter and/or a relatively similar cross sectional geometry along its length. In other embodiments, shaft 110 comprises a varying diameter along its length and/or a varying cross sectional geometry along its length. In some embodiments, shaft 110 comprises a first diameter on one end, and a different diameter on the opposite end, such as when both ends of the shaft are configured for insertion into a patient (referred to as a “double-ended” shaft). In these embodiments, either or both ends of shaft 110 comprise handle 150, or medical tool 100 does not include handle 150. In some embodiments shaft 110 comprises a non-linear construction, such as a curved or other non-linear construction, such as is described herebelow in reference to FIG. 9. Shaft 110 can comprise a rigid shaft, a flexible shaft, a malleable shaft, or it can comprise rigid, flexible and/or malleable portions. Shaft 110 can comprise a length of at least 3 inches, such as length of at least 6 inches or a length of approximately 9 inches (e.g. when medical tool 100 is constructed and arranged as a uterine sound). In some embodiments, shaft 110 comprises a length of at least 24 inches, such as when medical tool 100 comprises a laparoscopic tool with a shaft 110 comprising a length of at least 24 inches, at least 36 inches or at least 48 inches. Shaft 110 can comprise a diameter (e.g. comprise at least a portion with a diameter) of at least 1.5 mm, such as least 2.5 mm. In some embodiments, shaft 110 comprises a diameter between 1.5 mm and 25 mm, such as between 2.5 mm and 10 mm.

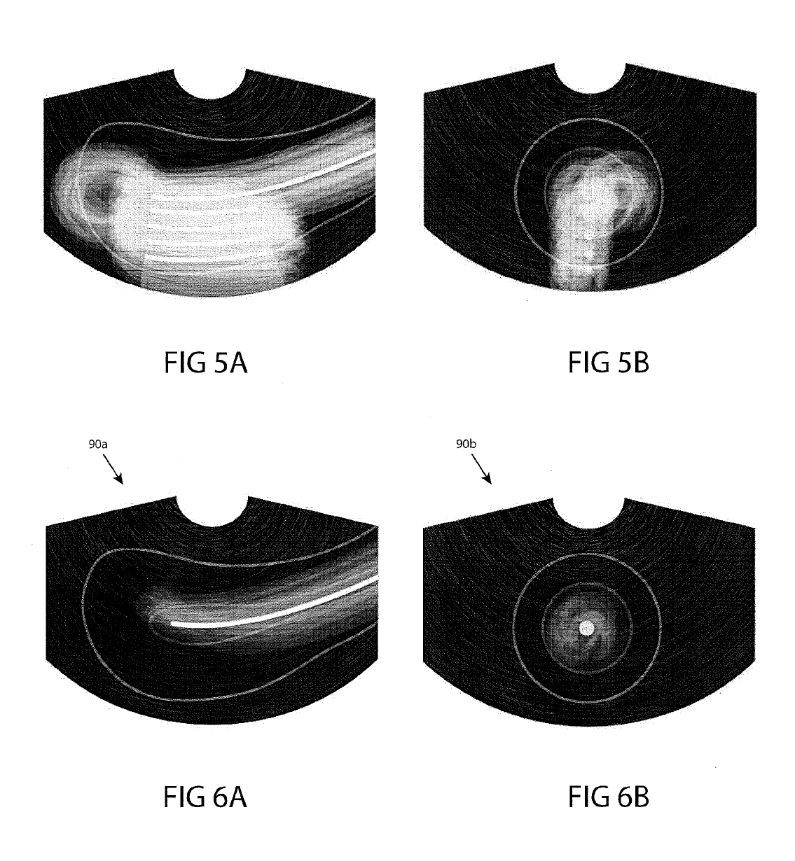

Shaft 110 can comprise a geometry constructed and arranged to provide shaft orientation information (e.g. translational and/or rotational information) on the medical image. In some embodiments, shaft 110 comprises multiple cross sectional geometries whose relative position provides orientation information on the medical image (e.g. medical image 90 described herebelow in reference to FIG. 5A-B or 6A-B). For example, a portion of wall 120 can comprise a first cross sectional geometry (e.g. a circular cross sectional geometry) and a corresponding (internal) portion of inner core 130 can comprise a second, different cross sectional geometry (e.g. a non-circular cross sectional geometry). Alternatively or additionally, shaft 110 can comprise one or more orientation markers, such as orientation marker 114 shown. Orientation marker 114 can comprise one or more visualizable markers (e.g. visualizable in the medical image produced by imaging device 50) selected from the group consisting of: ultrasonically reflective marker; radiopaque marker; magnetic marker; and combinations thereof. Orientation marker 114 can be positioned along a partial circumferential portion of wall 120 and/or inner core 130, such as to provide both axial (e.g. longitudinal) as well as rotational (e.g. angular) position information of medical tool 100 in medical image 90.

In some embodiments, shaft 110 comprises one or more lumens, not shown in FIG. 1 but such as lumens 117a and 117b described herebelow in reference to FIG. 3F, or lumen 117 described herebelow in reference to FIG. 3G. The one or more lumens can be arranged in a pattern selected from the group consisting of: concentric; non-concentric; adjacent; overlapping; non-overlapping; and combinations of one or more of these. The one or more lumens can be positioned within wall 120, within inner core 130 and/or between wall 120 and inner core 130. Shaft 110 can comprise one or more filaments, not shown in FIG. 1 but such as filament 118 described herebelow in reference to FIG. 3G. The filament can be positioned within wall 120, within inner core 130, between wall 120 and inner core 130 and/or within a lumen of shaft 110. The filament can comprise a filament selected from the group consisting of: electrical wire; control rod; optical fiber; fluid delivery tube, polymer, elastomer; and combinations of one or more of these. The filament can comprise a translatable filament, such as a control rod that can be advanced and/or retracted within shaft 110 such as to manipulate functional element 160 and/or another mechanism of medical tool 100. In some embodiments, inner core 130 comprises the filament, such as is described herebelow in reference to FIG. 3D. An inner core 130 comprising a filament can be operably attached to a functional element (e.g. functional element 160 shown in FIG. 1 and described herebelow), such as to activate or otherwise control the functional element.

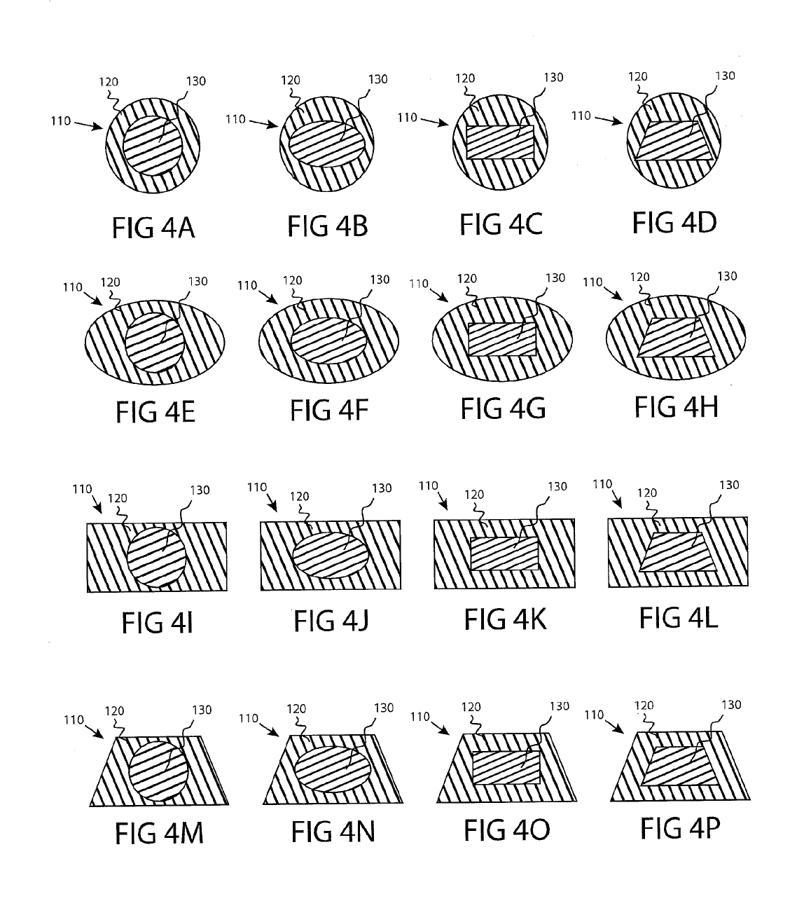

Wall 120 comprises an outer surface along its length. Wall 120 can comprise one or more hollowed segments along one or more portions of its length (e.g. such that an associated one or more inner cores 130 comprise or are positioned within the one or more hollowed segments of wall 120 as described herebelow in reference to FIG. 2A or 2B). Alternatively, wall 120 can comprise a single hollow segment along its entire length (e.g. when wall 120 comprises a tubular construction and inner core 130 comprises or is positioned within the single hollow segment of wall 120). The outer surface of wall 120 can comprise one or more cross sectional geometries, and an inner surface of wall 120 can comprise one or more similar or dissimilar cross sectional geometries, such as are described herebelow in detail in reference to FIGS. 4A-P. Cross sectional geometries of the outer and/or an inner surface of wall 120 can comprise a relatively continuous cross sectional geometry or a varying (e.g. tapered) cross sectional geometry along its length. An outer surface and/or inner surface of wall 120 can each comprise a cross sectional geometry selected from the group consisting of: circular; elliptical; rectangular; trapezoidal; regular polygon; irregular polygon; spiral; and combinations of one or more of these. In some embodiments, at least a portion of the outer surface of wall 120 comprises a circular shaped cross sectional geometry, and at least a portion of a corresponding inner surface of wall 120 comprises a cross sectional geometry selected from the group consisting of: circular; elliptical; rectangular; trapezoidal; regular polygon; irregular polygon; spiral; and combinations of one or more of these. In some embodiments, at least a portion of the outer surface of wall 120 comprises an elliptical shaped cross sectional geometry, and at least a portion of a corresponding inner surface of wall 120 comprises a cross sectional geometry selected from the group consisting of: circular; elliptical; rectangular; trapezoidal; regular polygon; irregular polygon; spiral; and combinations of one or more of these. In some embodiments, at least a portion of the outer surface of wall 120 comprises a rectangular shaped cross sectional geometry, and at least a portion of a corresponding inner surface of wall 120 comprises a cross sectional geometry selected from the group consisting of: circular; elliptical; rectangular; trapezoidal; regular polygon; irregular polygon; spiral; and combinations of one or more of these. In some embodiments, at least a portion of the outer surface of wall 120 comprises a trapezoidal shaped cross sectional geometry, and at least a portion of a corresponding inner surface of wall 120 comprises a cross sectional geometry selected from the group consisting of: circular; elliptical; rectangular; trapezoidal; regular polygon; irregular polygon; spiral; and combinations of one or more of these. In some embodiments, wall 120 comprises at least one portion of an outer surface with a cross sectional geometry that is different than the cross sectional geometry of at least one portion of an inner surface of wall 120 (e.g. inner and outer surfaces at the same relative axial location of wall 120).

In some embodiments, a portion of an inner surface of wall 120 defines an outer surface of inner core 130 (i.e. minimal or no gap between wall 120 and inner core 130 at that location). As stated above, an outer surface and/or inner surface of wall 120 can comprise a relatively continuous diameter and/or a relatively constant cross sectional geometry along the length of wall 120. Alternatively or additionally, an outer surface and/or inner surface of wall 120 can comprise a varying diameter and/or a varying cross sectional geometry along the length of wall 120. Similarly, an outer surface of inner core 130 can comprise a relatively continuous diameter and/or a relatively constant cross sectional geometry along the length of inner core 130, and/or an outer surface of inner core 130 can comprise a varying diameter (e.g. tapered) and/or varying cross sectional geometry. Inner core 130 can comprise an outer surface with a cross sectional geometry selected from the group consisting of: circular; elliptical; rectangular; trapezoidal; regular polygon; irregular polygon; spiral; and combinations of one or more of these.

Wall 120, inner core 130 and/or another component of shaft 110 can comprise one or more coatings, such as coating 121 positioned on shaft 110. Coating 121 can be positioned on an outer and/or inner surface of wall 120. Coating 121 can comprise an ultrasonically reflective coating; a radiopaque coating and/or a magnetic (e.g. magnetic or magnetizable) coating.

Medical tool 100 can comprise a tool constructed and arranged to perform a therapy and/or a tool constructed and arranged to gather patient information (e.g. patient anatomical or physiologic information). In some embodiments, medical tool 100 comprises a tool selected from the group consisting of: diagnostic tool; uterine sound (e.g. as described herebelow in reference to FIG. 3A); dilator; scissors (e.g. as described herebelow in reference to FIG. 3C); probe; manipulator; curette; forceps; tissue scraper (e.g. as described herebelow in reference to FIG. 3B); tissue grasping tool (e.g. as described herebelow in reference to FIG. 3D); tunneling tool; stylet; brachytherapy tool; radioactive source placement tool; electrical energy delivery device (e.g. a radiofrequency dessicator or cautery device); suction device; a robotically assisted tool (e.g. a tool connectable to a robot and/or a tool including one or more robotically controlled portions); and combinations of one or more of these.

Medical tool 100 can comprise a sterile tool and/or it can be constructed and arranged to be sterilized. In some embodiments, medical tool 100 is constructed and arranged to be sterilized multiple times (e.g. after one or more clinical uses). Medical tool 100 can be constructed and arranged for a single use (i.e. a single-use tool to be disposed of after use in one patient in a single clinical procedure). Alternatively, medical tool 100 can be constructed and arranged to be used in multiple procedures (e.g. to be used in a first patient, cleaned and re-sterilized and used in at least a second patient).

Medical tool 100 can comprise one or more functional elements, such as functional element 160 shown. Functional element 160 can comprise one or more sensors, transducers, a tip (e.g. an atraumatic tip) and/or other functional elements. Functional element 160 can comprise one or more functional elements positioned in, on (e.g. on an outer surface of) and/or within shaft 110, such as positioned on distal end 112 of shaft 110 as shown. In some embodiments, functional element 160 comprises a probe tip, such as when medical tool 100 comprises a uterine sound or is otherwise constructed and arranged to engage and/or identify a tissue surface such as a wall of the uterus of a patient. Functional element 160 can comprise a blunt (i.e. atraumatic tip), or a sharpened tip, such as a tip configured to tunnel through tissue (e.g. in a catheter or lead placement procedure). Functional element 160 can comprise an energy delivery element, such as an energy delivery element operably connected to console 70 (described herebelow) and configured to deliver energy selected from the group consisting of: electromagnetic energy; radiofrequency energy; microwave energy; light energy; laser light energy; sound energy; ultrasonic sound energy; subsonic sound energy; thermal energy; heat energy; cryogenic energy; infrared energy; terahertz energy; ultraviolet energy; visible light energy; and combinations thereof. Functional element 160 can comprise an agent delivery element (e.g. a needle, an iontophoretic element, a port and/or a catheter opening), such as an element configured to deliver one or more pharmaceutical drugs. Functional element 160 can comprise a radiation delivery element, such as an element configured to house a radioactive source, such as a radioactive source that is introduced into functional element 160 via one or more lumens of shaft 110 as described hereabove. Functional element 160 can comprise a radiation delivery element constructed and arranged to deliver a radiation source selected from the group consisting of: radioactive seeds; radionuclide agent; and combinations thereof. In some embodiments, functional element 160 comprises an element selected from the group consisting of: probe; tunneling tip; grasper; cutter; agent delivery element; electrode; energy delivery element; RF energy delivery element; cautery element; desiccating element; and combinations of one or more of these. In some embodiments, functional element 160 comprises an energy delivery element constructed and arranged to deliver an agent selected from the group consisting of: a drug; a chemotherapeutic agent; an agent configured for biologically targeted therapy; an immunotherapy agent; and combinations of one or more of these.

Medical tool 100 can comprise a handle, such as handle 150 shown positioned on the proximal end 111 of shaft 110. Handle 150 can comprise one or more controls, such as control 151 shown. Control 151 can be constructed and arranged to modify the shape of shaft 110, such as when control 151 is connected to a pull wire configured to deflect distal portion 113 of shaft 110. Control 151 can comprise a control configured to activate or otherwise modify the function of the one or more functional elements 160 of medical tool 100, such as when control 151 comprises a control selected from the group consisting of: switch; cam; mechanical linkage control; solenoid; and combinations of one or more of these.

System 10 can further comprise console 70, shown in FIG. 1 operably attached to handle 150 of medical tool 100. Console 70 can comprise an energy delivery unit, such as to provide one or more forms of energy to functional element 160 as described hereabove. Console 70 can comprise a camera or other imaging device, such as when functional element 160 comprises a camera, lens or other optical imaging component such as is described herebelow in reference to FIG. 3E. Console 70 can comprise one or more displays, such as a display configured to display one or more of: medical image 90; another image; console 70 information; medical tool 100 information; patient anatomic information; other patient information; and/or combinations of one or more of these. Console 70 can comprise a mechanical linkage control assembly, such as to robotically or otherwise control functional element 160, such as when functional element 160 comprises scissors, a grasper (as shown in FIG. 3D) and/or another mechanically manipulatable tool. Console 70 can comprise a user interface, not shown but such as an interface comprising one or more user input or user output components selected from the group consisting of: keyboard; keypad; mouse; screen; touchscreen; audio transducer such as a speaker or buzzer; and combinations of one or more of these.

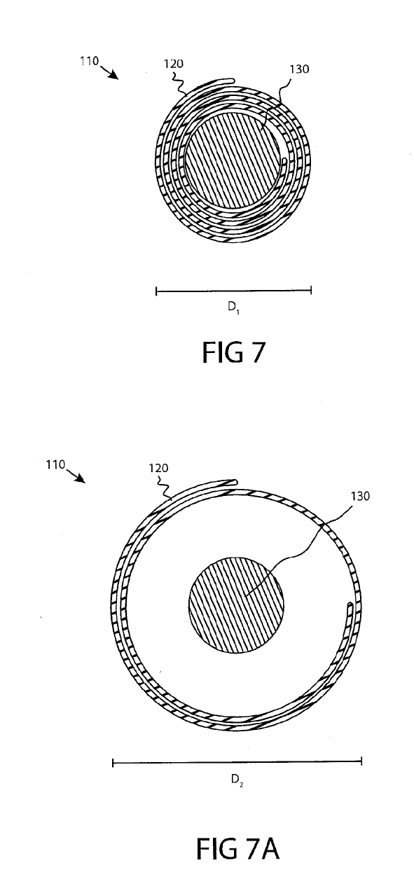

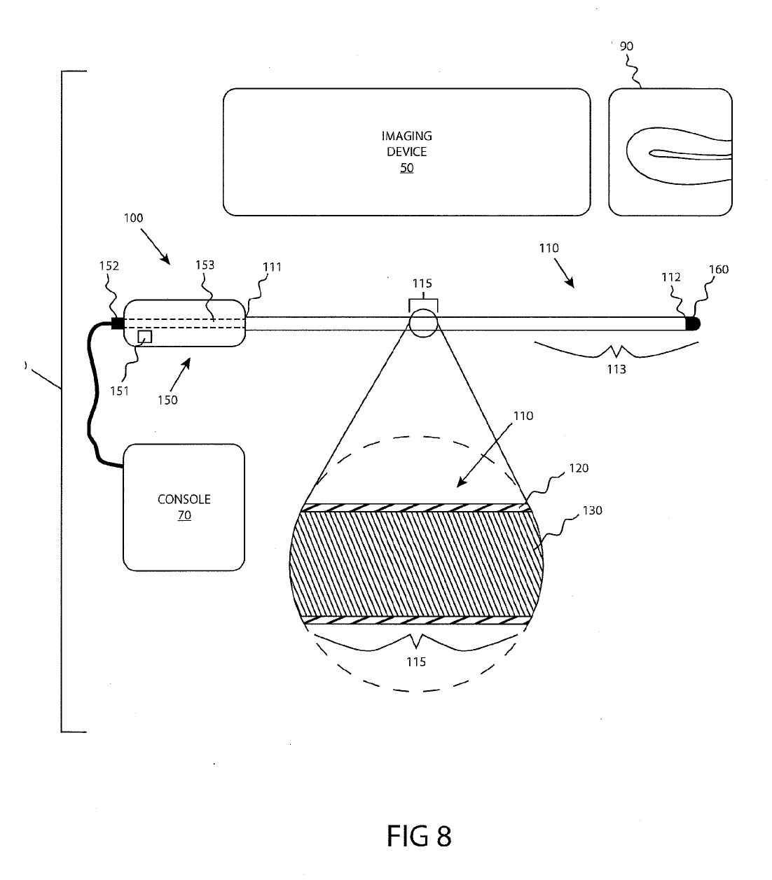

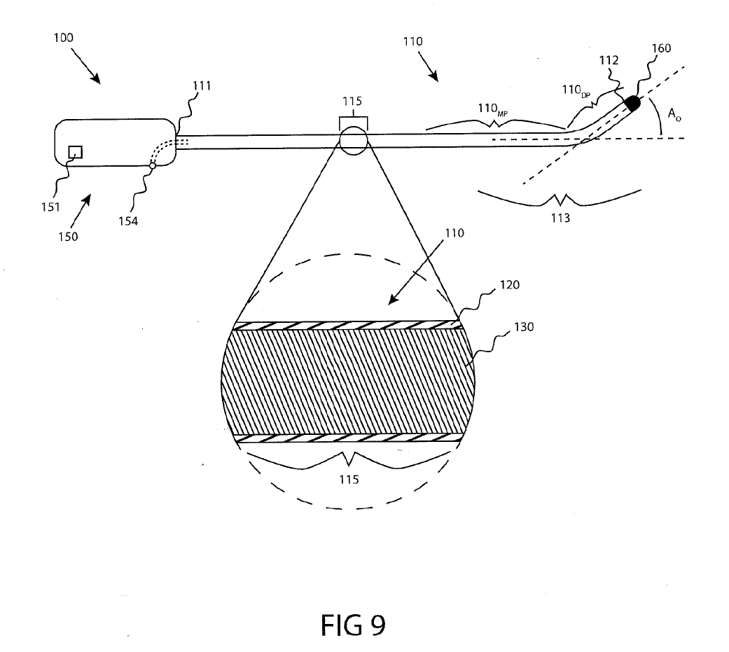

In some embodiments, wall 120 and/or inner core 130 can be constructed and arranged to be adjusted, such as to modify an image enhancing feature of wall 120 and/or inner core 130, respectively. In some embodiments, both wall 120 and inner core 130 are adjustable (e.g. each comprise adjustable pressures, densities, material and or diameter). For example, as described herebelow in reference to wall 120 of FIG. 7, wall 120 can be constructed and arranged to have an adjustable diameter or other adjustable parameter that can be adjusted by an operator (e.g. a clinician) during use, such as while being visualized (i.e. in real time) by imaging device 50. Alternatively or additionally, as described herebelow in reference to inner core 130 of FIG. 8, inner core 130 can be constructed and arranged to have an adjustable parameter selected from the group consisting of: pressure; diameter; type of material; mix of one or more materials; volume of material; density of material; and/or combinations of one or more of these. In some embodiments, these or other parameters are constructed and arranged to be adjusted by an operator (e.g. a clinician) during use.

In some embodiments, adjustment of wall 120 and/or inner core 130 is performed while being visualized (i.e. in real time) by imaging device 50. In some embodiments, wall 120 and/or inner core 130 are adjusted to: reduce distortion caused by medical tool 100; reduce scatter caused by medical tool 100; reduce noise caused by medical tool 100; and/or reduce degradation caused by medical tool 100 (e.g. to improve visualization of tissue in medical image 90). Alternatively or additionally, wall 120 and/or inner core 130 are adjusted to enhance visualization of medical tool 100 (e.g. enhance visualization of medical tool 100 in medical image 90). In some embodiments, wall 120 and/or inner core 130 are adjusted while multiple images 90 are created (e.g. in the creation of a video), such as to determine a desired level of adjustment for wall 120 and/or inner core 130, respectively.

In some embodiments, wall 120 and/or inner core 130 are constructed and arranged to be adjusted (e.g. manually and/or automatically) by console 70. For example, console 70 can be configured to furl or unfurl shaft 110, such as a furled shaft 110 as described herebelow in reference to FIG. 7. Alternatively or additionally, console 70 can be configured to adjust inner core 130, such as: to add or remove a material from inner core 130; to adjust the pressure of inner core 130; to adjust the density of inner core 130; to change one or more materials of inner core 130; and combinations of one or more of these.

In some embodiments, system 10 comprises multiple medical tools 100, in a “kit” configuration. For example, system 10 can comprises multiple medical tools 100 with similar or dissimilar characteristics, such as similar or dissimilar shaft 110 diameters and/or wall 120 thicknesses. In some embodiments, system 10 comprises at least two medical tools 100 with similar diameters of shaft 110 but different wall 120 thicknesses, such as to have a different effect on medical image 90.

In some embodiments, system 10 comprises a kit of multiple medical tools 100, each including a shaft 110 with a diameter (at least the diameter of shaft 110 at and/or proximate distal end 112) of approximately 1.5 mm; 2.0 mm; 2.5 mm; 5.0 mm; 7.5 mm; 10.0 mm and/or 12.5 mm. In some embodiments, system 10 comprises a kit of multiple medical tools 100, each including a shaft 110 including two diameters selected from the group consisting of: 1.5 mm; 2.0 mm; 2.5 mm; 5.0 mm; 7.5 mm; 10.0 mm; and 12.5 mm, such as when each medical tool 100 comprises a double-ended configuration as described herein. In some embodiments, system 10 comprises a kit of multiple medical tools 100, each including a diameter ranging from 1.5 mm to 6.0 mm, 2.0 mm to 5.0 mm, or 3.0 mm to 5.0 mm. For example, system 10 can comprise a kit of three medical tools 100 with shaft 110 diameters of 3.0 mm, 4.0 mm and 5.0 mm (or a set of less than three tools where one or more of the tools comprises a double-ended shaft). System 10 can comprise a kit of five medical tools 100 with shaft 110 diameters of 2.0 mm, 3.0 mm, 4.0 mm, 5.0 mm and 6.0 mm (or a set of less than five tools where one or more of the tools comprises a double-ended shaft). One or more of the medical tools 100 of a kit of system 10 can comprise a double-ended shaft 110 configuration. One or more of the medical tools 100 of a kit of system 10 can comprise an attachable handle (e.g. attached by engaging threads).

Referring now to FIG. 2A, a sectional side view of the distal portion of a medical tool comprising a functional element at its tip and three image enhancing features are illustrated, consistent with the present inventive concepts. Shaft 110 comprises wall 120 which surrounds multiple inner cores 130, inner core 130a shown positioned in segment 115a, inner core 130b shown positioned in segment 115b, and inner core 130c shown positioned in segment 115c. Any of inner cores 130a-c (singly or collectively inner core 130) can be constructed and arranged to enhance a medical image as described hereabove. Segments 115a-c (singly or collectively segment 115) can be positioned in distal portion 113 of shaft 110. Alternatively, one or more of segments 115 can be positioned in a different (e.g. more proximal) portion of shaft 110. Positioned on the distal end 112 of shaft 110 is functional element 160. Functional element 160 can be operably connected to a filament (e.g. a wire, optical fiber and/or translating cable) such as when inner core 130 comprises a translating or non-translating filament operably connected to functional element 160 as described herebelow in reference to FIG. 3D, or such as when shaft 110 surrounds a separate, translating or non-translating filament operably connected to functional element 160, such as filament 118 described herebelow in reference to FIG. 3G. Shaft 110, wall 120, inner cores 130 and/or functional element 160 can be of similar construction and arrangement to the similar components described hereabove in reference to FIG. 1. Inner core 130c is positioned both within wall 120 and within functional element 160, such as to enhance an image comprising functional element 160 in addition to enhancing an image comprising shaft 110.

Referring now to FIG. 2B, a sectional side view of the distal portion of a medical tool comprising a functional element at its tip and four image enhancing features are illustrated, consistent with the present inventive concepts. Shaft 110 comprises wall 120 which surrounds multiple inner cores 130, inner core 130a shown positioned in segment 115a, inner core 130b shown positioned in segment 115b, and inner core 130c shown positioned in segment 115c. Segments 115a-c (singly or collectively segment 115) can be positioned in distal portion 113 of shaft 110. Alternatively, one or more of segments 115 can be positioned in a different (e.g. more proximal) portion of shaft 110. Positioned on the distal end 112 of shaft 110 is functional element 160. Functional element 160 can be operably connected to a filament (e.g. a wire, optical fiber and/or translating cable) such as when inner core 130 comprises a translating or non-translating filament operably connected to functional element 160 as described herebelow in reference to FIG. 3D, or such as when shaft 110 surrounds a separate, translating or non-translating filament operably connected to functional element 160, such as filament 118 described herebelow in reference to FIG. 3G. Positioned within functional element 160, at segment 115d, is inner core 130d. Any of inner cores 130a-d (singly or collectively inner core 130) can be constructed and arranged to enhance a medical image as described hereabove (e.g. to enhance an image comprising shaft 110 and/or functional element 160). Shaft 110, wall 120, inner cores 130 and/or functional element 160 can be of similar construction and arrangement to the similar components described hereabove in reference to FIG. 1.

Referring now to FIG. 3A, a side view and a magnified side sectional view of a uterine sound tool comprising an image enhancing feature are illustrated, consistent with the present inventive concepts. Medical tool 100 of FIG. 3A is constructed and arranged as a uterine sound and/or is otherwise configured to measure and/or probe the surface of a hollow organ or conduit of a patient. Medical tool 100 comprises shaft 110 comprising proximal end 111, distal portion 113 and distal end 112. A handle 150, comprising control 151, is positioned on proximal end 111 of shaft 110. Functional element 160 is positioned on the distal end 112 of shaft 110. Functional element 160 can comprise a probe and/or a blunt or otherwise atraumatic tip configured to engage tissue, such as to provide tactile feedback of the tissue engagement to an operator (e.g. via shaft 110 and handle 150). Medical tool 100 of FIG. 3A can be constructed and arranged to measure the length and direction of the cervical canal and uterus, to manipulate the cervix and/or the uterus, to determine the level of dilation and/or to induce further dilation. Shaft 110 can comprise ruler or other dimensional markings, such as ruler 116 shown.

Shaft 110 further comprises wall 120 and inner core 130, as shown in the magnified view of segment 115 of shaft 110. Shaft 110, wall 120, inner core 130, functional element 160, handle 150 and/or control 151 can be of similar construction and arrangement to the similar components described hereabove in reference to FIG. 1. Handle can comprise a port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1. Wall 120 and/or inner core 130 can comprise one or more malleable or non-malleable materials, and can comprise a variable length (e.g. telescopic) and/or a varying diameter.

Referring now to FIG. 3B, a side view and a magnified side sectional view of a scraping tool comprising an image enhancing feature are illustrated, consistent with the present inventive concepts. Medical tool 100 of FIG. 3B is constructed and arranged as a tissue scraper and/or is otherwise configured to scrape, cut, abrade and/or debride living or dead tissue of a patient. Medical tool 100 comprises shaft 110 comprising proximal end 111 and distal end 112. A handle 150 is positioned on proximal end 111 of shaft 110. Functional element 160 is positioned on the distal end 112 of shaft 110. Functional element 160 comprises an element configured to scrape, cut, abrade and/or debride living or dead tissue.

Shaft 110 further comprises wall 120 and inner core 130, as shown in the magnified view of segment 115 of shaft 110. Shaft 110, wall 120, inner core 130, functional element 160 and/or handle 150 can be of similar construction and arrangement to the similar components described hereabove in reference to FIG. 1. Handle can comprise a port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 3C, a side view and a magnified side sectional view of forceps comprising an image enhancing feature are illustrated, consistent with the present inventive concepts. Medical tool 100 of FIG. 3C is constructed and arranged as a forceps device and/or is otherwise configured to engage tissue of a patient. Medical tool 100 comprises shaft 110, comprising a first shaft 110a and a second shaft 110b, rotatably connected at hinge 125. A handle 150 comprising finger loops is positioned on proximal ends 111a and 111b of shafts 110a and 110b, respectively. Functional element 160 comprises two tissue engaging elements positioned on distal ends 112a and 112b of shafts 110a and 110b, respectively.

Shaft 110 further comprises wall 120 and inner core 130, such as wall 120b and inner core 130b shown in the magnified view of segment 115 of shaft 110b. Shaft 110, wall 120, inner core 130, functional element 160 and/or handle 150 can be of similar construction and arrangement to the similar components described hereabove in reference to FIG. 1. Handle can comprise a port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 3D, a side view and a magnified side sectional view of a tissue grasping tool comprising an image enhancing feature and a translating inner portion are illustrated, consistent with the present inventive concepts. Medical tool 100 of FIG. 3D is constructed and arranged as scissors and/or is otherwise configured to cut tissue of a patient. Medical tool 100 comprises shaft 110 comprising proximal end 111 and distal end 112. A handle 150, comprising control 151, is positioned on proximal end 111 of shaft 110. Functional element 160 is positioned on the distal end 112 of shaft 110. Functional element 160 comprises a rotating scissor element and control 151 comprises a trigger mechanism operably connected to functional element 160, such as via a translating inner core 130 constructed and arranged to be advanced and/or retracted to cause the scissor-based functional element 160 to operate.

Shaft 110 further comprises wall 120 and inner core 130, as shown in the magnified view of segment 115 of shaft 110. Shaft 110, wall 120, inner core 130, functional element 160, handle 150 and/or control 151 can be of similar construction and arrangement to the similar components described hereabove in reference to FIG. 1. Handle can comprise a port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 3E, a schematic view and a magnified side sectional view of a system including a medical tool comprising an image enhancing feature and a camera is illustrated, consistent with the present inventive concepts. Medical tool 100 of FIG. 3E is constructed and arranged as a medical tool including a camera and/or is otherwise configured to create a medical image. Medical tool 100 comprises shaft 110 comprising proximal end 111, distal portion 113 and distal end 112. A handle 150, comprising control 151, is positioned on proximal end 111 of shaft 110. Functional element 160 is positioned on the distal end 112 of shaft 110. Positioned along shaft 110 (e.g. in distal portion 113 as shown) is a second functional element, optical element 160′. Optical element 160′ can comprise one or more elements configured to produce a medical image (e.g. an image correlating to a field of view along the axis of shaft 110 as shown), such as an element selected from the group consisting of: a camera; a lens; a prism; and combinations of one or more of these. Optical element 160′ is operably attached to the distal end of filament 118, which travels proximally to handle 150. In some embodiments, filament 118 (e.g. via one or more electrical, optical or other connectors) operably attaches to console 70. Filament 118 can comprise an element selected from the group consisting of: one or more wires such as one or more electrical wires; one or more optical fibers; one or more control rods; one or more fluid delivery tubes; and combinations of one or more of these. In some embodiments, optical element 160′ comprises a lens, prism or other optical component which operably attaches to the distal end of an optical fiber-based filament 118, which in turn operably attaches on its proximal end to a camera device within handle 150 and/or console 70. In some embodiments, optical element 160′ comprises a camera or other image capturing element which operably attaches to the distal end of an electrical wire-based filament 118, which in turn operably attaches on its proximal end to an image-receiving and/or image-displaying element of handle 150 and/or console 70. Control 151 can be configured to activate or otherwise modify the function of optical element 160′ and/or any components operably attached thereto.

Shaft 110 further comprises wall 120 and inner core 130, as shown in the magnified view of segment 115 of shaft 110. Shaft 110, wall 120, inner core 130, functional element 160, handle 150, control 151, and/or console 70 can be of similar construction and arrangement to the similar components described hereabove in reference to FIG. 1. In some embodiments, wall 120 comprises a metal material and inner core 130 comprises a non-metallic solid, a fluid (e.g. a liquid and/or a gas) and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 3F, a side view, a magnified side sectional view and a magnified end sectional view of a medical tool comprising an image enhancing feature and a dual lumen shaft is illustrated, consistent with the present inventive concepts. Medical tool 100 of FIG. 3F is constructed and arranged as a medical tool comprising multiple lumens (e.g. the two “D-shaped” lumens 117a and 117b shown in FIG. 3F). Medical tool 100 comprises shaft 110 comprising proximal end 111, distal portion 113 and distal end 112. A handle 150, comprising control 151, is positioned on proximal end 111 of shaft 110. Functional element 160 is positioned on the distal end 112 of shaft 110. Shaft 110 comprises lumens 117a and 117b, into which inner cores 130a and 130b, respectively, have been positioned. In some embodiments, inner cores 130a and/or 130b (singly or collectively inner core 130) comprise a translating core, as described hereabove in reference to FIG. 1 or FIG. 3D. In some embodiments, inner core 130a and/or 130b is removable from shaft 110, as described hereabove in reference to FIG. 1. In some embodiments, inner core 130a comprises a first material, and inner core 130b comprises a second material, different than the first material. In some embodiments, inner core 130a comprises a gas or a vacuum, and inner core 130b comprises a liquid or solid material. In some embodiments, inner core 130a comprises a malleable material.

Lumens 117a and/or 117b can each comprise cross sections comprising a geometry selected from the group consisting of: circle; oval; rectangle; trapezoid; regular polygon; irregular polygon; spiral; and combinations of one or more of these. Lumens 117a and/or 117b can comprise relatively adjacent cross sections or cross sections separated by a gap. Lumens 117a and 117b can comprise concentric or non-concentric rings. The cross sections of lumens 117a and/or 117b can vary along the length of shaft 110.

Shaft 110 further comprises wall 120 which surrounds the inner cores 130, as shown in the magnified side sectional view and end sectional views of segment 115 of shaft 110. Shaft 110, wall 120, inner cores 130, functional element 160, handle 150 and/or control 151 can be of similar construction and arrangement to the similar components described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 3G, a side view, a magnified side sectional view and a magnified end sectional view of a medical tool comprising an image enhancing feature and a translating shaft is illustrated, consistent with the present inventive concepts. Medical tool 100 of FIG. 3G comprises a tool with a translating filament 118 positioned in a shaft 110, such as a translating filament 118 operably attached to a functional element 160. Medical tool 100 comprises shaft 110 comprising proximal end 111, distal portion 113 and distal end 112. A handle 150, comprising control 151, is positioned on proximal end 111 of shaft 110. Functional element 160 is positioned on the distal end 112 of shaft 110. Positioned within shaft 110 is lumen 117 into which filament 118 is positioned. Inner core 130 can comprise the donut shape shown, such that lumen 117 concentrically passes through inner core 130. In some embodiments, lumen 117 comprises multiple lumens that pass through one or more of: within inner core 130; within wall 120; and/or within a space between wall 120 and inner core 130. Filament 118 can be constructed and arranged to be translated within lumen 117, such as when filament 118 comprises a control cable configured to deploy, actuate and/or otherwise control functional element 160. In some embodiments, filament 118 comprises a stationary filament, such as a filament 118 comprising one or more filaments selected from the group consisting of: one or more wires such as one or more wires electrically attached to an electrically-based functional element 160 such as a radiofrequency energy-delivering electrode; one or more fluid delivery tubes (e.g. drug or other agent delivery tubes, hydraulic fluid delivery tubes, pneumatic fluid delivery tubes, and the like); one or more optical fibers such as when functional element 160 comprises a laser or other light delivery element and/or a camera; and combinations of one or more of these.

Lumen 117 and/or filament 118 can each comprise cross sections comprising a geometry selected from the group consisting of: circle; oval; rectangle; trapezoid; regular polygon; irregular polygon; spiral; and combinations of one or more of these. Filament 118 can be positioned concentrically within lumen 117 or at an eccentric location. The cross sections of lumen 117 and/or filament 118 can vary along the length of shaft 110.

Shaft 110 further comprises wall 120 and inner core 130, as shown in the magnified view of segment 115 of shaft 110. Shaft 110, wall 120, inner core 130, functional element 160, handle 150 and/or control 151 can be of similar construction and arrangement to the similar components described hereabove in reference to FIG. 1. Handle 150 can comprise a port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIGS. 4A-4P, end sectional views of various medical tool shafts comprising different cross sectional geometries constructed and arranged to enhance an image are illustrated, consistent with the present inventive concepts. FIGS. 4A-4P illustrate various cross sections for one or more segments of shaft 110 (e.g. segments 115 described herein) that include both wall 120 and inner core 130. In FIG. 4A, a segment of shaft 110 comprises a wall 120 with an outer surface with a circular cross sectional geometry and an inner core 130 with an outer surface with a circular cross sectional geometry. In FIG. 4B, a segment of shaft 110 comprises a wall 120 with an outer surface with a circular cross sectional geometry and an inner core 130 with an outer surface with an elliptical cross sectional geometry. In FIG. 4C, a segment of shaft 110 comprises a wall 120 with an outer surface with a circular cross sectional geometry and an inner core 130 with an outer surface with a rectangular cross sectional geometry. In FIG. 4D, a segment of shaft 110 comprises a wall 120 with an outer surface with a circular cross sectional geometry and an inner core 130 with an outer surface with a trapezoidal cross sectional geometry.

In FIG. 4E, a segment of shaft 110 comprises a wall 120 with an outer surface with an elliptical cross sectional geometry and an inner core 130 with an outer surface with a circular cross sectional geometry. In FIG. 4F, a segment of shaft 110 comprises a wall 120 with an outer surface with an elliptical cross sectional geometry and an inner core 130 with an outer surface with an elliptical cross sectional geometry. In FIG. 4G, a segment of shaft 110 comprises a wall 120 with an outer surface with an elliptical cross sectional geometry and an inner core 130 with an outer surface with a rectangular cross sectional geometry. In FIG. 4H, a segment of shaft 110 comprises a wall 120 with an outer surface with an elliptical cross sectional geometry and an inner core 130 with an outer surface with a trapezoidal cross sectional geometry.

In FIG. 4I, a segment of shaft 110 comprises a wall 120 with an outer surface with a rectangular cross sectional geometry and an inner core 130 with an outer surface with a circular cross sectional geometry. In FIG. 4J, a segment of shaft 110 comprises a wall 120 with an outer surface with a rectangular cross sectional geometry and an inner core 130 with an outer surface with an elliptical cross sectional geometry. In FIG. 4K, a segment of shaft 110 comprises a wall 120 with an outer surface with a rectangular cross sectional geometry and an inner core 130 with an outer surface with a rectangular cross sectional geometry. In FIG. 4L, a segment of shaft 110 comprises a wall 120 with an outer surface with a rectangular cross sectional geometry and an inner core 130 with an outer surface with a trapezoidal cross sectional geometry.

In FIG. 4M, a segment of shaft 110 comprises a wall 120 with an outer surface with a trapezoidal cross sectional geometry and an inner core 130 with an outer surface with a circular cross sectional geometry. In FIG. 4J, a segment of shaft 110 comprises a wall 120 with an outer surface with a trapezoidal cross sectional geometry and an inner core 130 with an outer surface with an elliptical cross sectional geometry. In FIG. 4K, a segment of shaft 110 comprises a wall 120 with an outer surface with a trapezoidal cross sectional geometry and an inner core 130 with an outer surface with a rectangular cross sectional geometry. In FIG. 4L, a segment of shaft 110 comprises a wall 120 with an outer surface with a trapezoidal cross sectional geometry and an inner core 130 with an outer surface with a trapezoidal cross sectional geometry.

The cross sectional profiles of wall 120 and inner core 130 can be used in shaft 110 configurations including multiple walls 120 and/or multiple inner cores 130. The multiple walls 120 and/or multiple inner cores 130 can be arranged in adjacent, overlapping, non-overlapping, concentric and/or non-concentric patterns.

Referring now to FIGS. 5A and 5B, recreations of an ultrasound image, a sagittal view and an axial view, respectively, of the uterus of a patient into which a standard tool has been positioned are illustrated. In FIGS. 6A and 6B, medical image 90a and 90b are shown, respectively, comprising similar views of a uterus to FIGS. 5A and 5B, respectively, but the standard tool has been replaced with a medical tool of the present inventive concepts, the medical tool including one or more image enhancing features, such as have been described in detail hereabove.

Referring now to FIG. 7, an end sectional view of a shaft of a medical tool comprising an adjustable diameter wall is illustrated, consistent with the present inventive concepts. Shaft 110 comprises wall 120 which comprises a furled sheet of material, such as a furled sheet of stainless steel, titanium or nickel titanium alloy. A handle, not shown but such as handle 150 described hereabove, can be attached to wall 120 such that rotation of handle 110 in one direction causes wall 120 to unfurl (e.g. increase the diameter of shaft 110) and rotation of handle 150 in the opposite direction causes wall 120 to furl (e.g. to decrease the diameter of shaft 110). In some embodiments, inner core 130 is also adjustable (e.g. as described herebelow in reference to inner core 130 of FIG. 8), such as to adjust the diameter (e.g. by adjusting the pressure) of inner core 130 and correspondingly exert a force to adjust the diameter of wall 120. In some embodiments, the density of inner core 130 is adjusted (e.g. by adjusting the pressure).

Shaft 110, wall 120 and/or inner core 130 can be of similar construction and arrangement to the similar components described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

In FIG. 7A, shaft 110's diameter has been increased to a magnitude D₂, which is larger than the diameter D₁ of shaft 110 shown in FIG. 7. While the embodiments of FIGS. 7 and 7A illustrate a shaft 110 whose diameter is changing via furling or unfurling wall 120 comprising a coiled sheet (e.g. a coiled sheet of stainless steel or other metal), other configurations of shaft 110 and/or wall 120 can be included to allow changing of the diameter of shaft 110. For example, wall 120 can comprise: a stretchable tube that expands under tension; a segmented, hinged tubular structure which can be radially expanded or compacted; and the like.

Referring now to FIG. 8, a side view and a magnified side sectional view of a medical tool comprising an adjustable inner core is illustrated, consistent with the present inventive concepts. Medical tool 100 of FIG. 8 is constructed and arranged as a medical tool including an adjustable inner core 130. Medical tool 100 comprises shaft 110 comprising proximal end 111, distal portion 113 and distal end 112. A handle 150, comprising control 151, is positioned on proximal end 111 of shaft 110. Functional element 160 is positioned on the distal end 112 of shaft 110.

Shaft 110 further comprises wall 120 and inner core 130, as shown in the magnified view of segment 115 of shaft 110. In some embodiments, inner core 130 is fluidly attached to lumen 153 of handle 150, which in turn is fluidly attached to port 152. Console 70 can be fluidly attached to port 152, such that one or more parameters of inner core 130 can be adjusted by console 70, via port 152 and lumen 153. For example, the pressure within inner core 130 can be adjusted by console 70. Alternatively or additionally, one or more other parameters of inner core 130 can be adjusted such as an inner core 130 parameter selected from the group consisting of: pressure; diameter; type of material; mix of one or more materials; volume of material; density of material; and/or combinations of one or more of these. In some embodiments, these or other parameters of inner core 130 are adjusted by adding and/or removing material to and/or from inner core 130 via port 152 and lumen 153 of handle 150. In some embodiments, wall 120 is also adjustable, such as is described hereabove in reference to FIGS. 7 and 7A.

Shaft 110, wall 120, inner core 130, functional element 160, handle 150, control 151, and/or console 70 can be of similar construction and arrangement to the similar components described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1.

Referring now to FIG. 9, a side view and a magnified side sectional view of a medical tool comprising an adjustable image enhancing feature is illustrated, consistent with the present inventive concepts. Medical tool 100 comprises shaft 110 comprising proximal end 111, distal portion 113 and distal end 112. In some embodiments, medical tool 100 comprises a handle 150, such as a threaded or other attachable handle, or a pre-attached handle, positioned on proximal end 111 of shaft 110. Handle 150 can comprise a control, control 151 shown. Functional element 160 can be positioned on the distal end 112 of shaft 110. Functional element 160 can comprise a probe and/or a blunt or otherwise atraumatic tip configured to engage tissue, such as to provide tactile feedback of the tissue engagement to an operator (e.g. via shaft 110 and handle 150). Medical tool 100 of FIG. 9 can be constructed and arranged to measure the length and direction of the cervical canal and uterus, to manipulate the cervix and/or the uterus, to determine the level of dilation and/or to induce further dilation.

Shaft 110 further comprises wall 120 and inner core 130, as shown in the magnified view of segment 115 of shaft 110. Medical tool 100 of FIG. 9 comprises an access port, fill port 154, which provides access to inner core 130. Fill port 154 can comprise a luer connector or other access port configured to allow material to be introduced into and/or removed from shaft 110 (e.g. via a syringe or other device to position or remove inner core 130 from at least a segment of shaft 110). In some embodiments, medical tool 100 is shipped to a user with inner core 130 comprising gas or other removable material positioned within shaft 110. In these embodiments, inner core 130 can comprise air or other gas that is used in a clinical procedure in which medical tool 100 is imaged with ultrasound (e.g. imaging device 50 of FIG. 1 comprises an ultrasonic imager). Alternatively, a user (e.g. a clinician or other user) can introduce a non-gas into shaft 110 (e.g. a gel or other liquid, or a flowable solid material), such as to change inner core 130 to a liquid or solid material, after which medical tool 100 is imaged by MM (e.g. imaging device 50 of FIG. 1 comprises an MRI).

In some embodiments, shaft 110 comprises mid-portion 110_{MP} and distal portion 110_{DP}. Distal portion 110_{DP} can comprise an axis that is angularly offset from the axis of mid-portion 110_{MP}, such as an angular offset A_{O} between 5° and 60°.

Shaft 110, wall 120, inner core 130, functional element 160, handle 150 and/or control 151 can be of similar construction and arrangement to the similar components described hereabove in reference to FIG. 1. Handle 150 can comprise a second port or otherwise be attachable to a separate device, such as console 70 also described hereabove in reference to FIG. 1. In some embodiments, wall 120 comprises a metal material and inner core 130 comprises a non-metallic solid, a fluid and/or a vacuum. In some embodiments, wall 120 and/or inner core 130 comprise materials and/or dimensions (e.g. thicknesses) constructed and arranged to enhance a medical image, such as is described hereabove in reference to FIG. 1. Wall 120 and/or inner core 130 can comprise one or more malleable or non-malleable materials, and can comprise a variable length (e.g. telescopic) and/or a varying diameter.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1-234. (canceled)

235. A medical tool comprising:
an elongate shaft comprising a wall and at least one inner core positioned within the wall;
wherein the wall comprises metal with a thickness of less than or equal to 0.5 mm;
wherein the inner core comprises a fluid; and
wherein the medical tool is configured to perform a diagnostic procedure on the uterus of a patient.
